(19) United States
(12) Patent Application Publication
(10) Pub. No.: US 2019/0194100 A1
(43) Pub. Date: Jun. 27, 2019

(54) METHOD FOR THE PRODUCTION OF GLYCOLS FROM AN ANHYDROSUGAR FEED

(71) Applicant: SHELL OIL COMPANY, HOUSTON, TX (US)

(72) Inventors: Pen-Chung WANG, Houston, TX (US); Smita EDULJI, Houston, TX (US)

(21) Appl. No.: 16/327,068

(22) PCT Filed: Aug. 21, 2017
(86) PCT No.: PCT/US2017/047749

§ 371 Date: Feb. 21, 2019

Related U.S. Application Data
(60) Provisional application No. 62/378,257, filed on Aug. 23, 2016.

(51) Int. Cl. C07C 29/60; C07C 29/14; C07C 29/132

(52) U.S. Cl. CPC C07C 29/60; C07C 29/132; C07C 29/14

(57) ABSTRACT

Implementations of the disclosed subject matter provide a method for producing glycols from an anhydrosugar feed. The method may include contacting, in a reactor under hydrogenolysis conditions, the anhydrosugar feed with a bi-functional catalyst system. The bi-functional catalyst system may include a retro-Aldol catalyst and a hydrogenation catalyst. A product stream may be obtained from the first reactor including ethylene glycol and propylene glycol.

### CROSS-REFERENCE TO RELATED APPLICATIONS

This application claims priority to U.S. Provisional Application Ser. No. 62/378,257 filed Aug. 23, 2016, the entire disclosure of which is hereby incorporated by reference.

### TECHNICAL FIELD OF THE INVENTION

The present invention relates to a process for converting an anhydrosugar feed stock into glycols. More specifically the present invention relates to a process for preparing glycols, particularly ethylene glycol and propylene glycol, by converting an anhydrosugar feed stock material in a reactor using a bi-functional catalyst system.

### BACKGROUND

Glycols such as ethylene glycol and propylene glycol are valuable materials with a multitude of commercial applications, e.g. as heat transfer media, antifreeze, and precursors to polymers, such as PET. The market for ethylene and propylene glycols (EG and PG) is expanding worldwide, with the EG market being vastly bigger than the market for PG (i.e., 1,2-propylene glycol). Ethylene and propylene glycols are typically made on an industrial scale by hydrolysis of the corresponding alkylene oxides, which are the oxidation products of ethylene and propylene, produced from fossil fuels/petrochemical feed stocks involving multiple processing steps. Use of bio-based feed stocks for the production of energy and chemicals has become increasingly desirable in the industry since this approach to use feeds from renewable sources provides a pathway for sustainable development.

Anhydrosugars are a class of compounds that can be converted to sugars via a catalyzed chemical reaction with water in a process commonly referred to as hydrolysis. One way to produce anhydrosugars is to heat the biomass (e.g., the lignocellulose materials) to high temperatures under controlled conditions. The process is termed fast pyrolysis and various equipment has been employed to effect the thermal dissociation. Typically, the materials are heated rapidly in an inert gas, which carries off the vaporized products to be collected as a condensate after cooling the stream. The condensate oil (bio oil) from fast pyrolysis of lignocellulosic substrates contains considerable amounts of anhydrosugars in addition to other oxygenated components derived from the various biomass constituents. The anhydrosugars comprise a variety of mono-, di-, oligosaccharides containing an additional oxidic ring. One of the largest constituent of this family is levoglucosan or 1,6-D-anhydroglucose (as shown below). Anhydrosugars have the structural features resembling that of ethylene glycol; each carbon has one attached hydroxyl group or contains an oxygen function that can be readily converted into a hydroxyl. As such, EG and PG can be produced if the C—C bonds are selectively cleaved into C2 and C3 units.

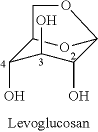 [SMILES: OC1C2COC(O2)C(O)C1O]

Several reviews of fast pyrolysis processes have been published. For example, see A. V. Bridgwater, D. Meier, and D. Radlein, 1999; S. Czernik and A. V. Bridgwater, 2004, Zhang Qi, Chang Jie, Wang Tiejun and Xu Ying, 2007; Kathlene Jacobson, Kalpana C. Maheria and Ajay Kumar Dalai, 2013.

As with many chemical processes, the reaction product stream in these processes comprises a number of desired materials as well as diluents, by-products and other undesirable materials. In order to provide a high value process, the desirable product or products must be obtainable from the reaction product stream in high purity with a high percentage recovery of each product and with as low as possible use of energy, chemical components and complex equipment.

Therefore, it would be advantageous to provide an improved method suitable for the production of glycols from anhydrosugar feeds in order to make the overall glycol production process more economical than processes disclosed previously in the industry.

### BRIEF SUMMARY

According to an embodiment of the disclosed subject matter, a method for producing glycols from an anhydrosugar feed may include contacting, in a reactor under hydrogenolysis conditions, the anhydrosugar feed with a bi-functional catalyst system. The bi-functional catalyst system may include a retro-Aldol catalyst and a hydrogenation catalyst. A product stream may be obtained from the reactor including ethylene glycol and propylene glycol.

Implementations of the disclosed subject matter provide an improved method for producing ethylene glycol from an anhydrosugar feed. The disclosed subject matter allows the desirable products of EG and PG to be obtained from the reaction product stream in high purity with a high percentage recovery of each product and with relatively low use of energy, chemical components and complex equipment as compared to prior processes. This method results in a production of glycols from anhydrosugar feeds that makes the overall glycol production process more economical than processes disclosed previously in the industry. Additional features, advantages, and embodiments of the disclosed subject matter may be set forth or apparent from consideration of the following detailed description, drawings, and claims. Moreover, it is to be understood that both the foregoing summary and the following detailed description are examples and are intended to provide further explanation without limiting the scope of the claims.

### BRIEF DESCRIPTION OF THE DRAWINGS

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

FIG. 1 shows an example process scheme according to an implementation of the disclosed subject matter.

### DETAILED DESCRIPTION

Renewable bio-mass feeds are readily available and can be converted to bio-oil after fast pyrolysis. This bio-oil includes multiple components including water, lignin, acid, ketone aldehyde, and anhydrosugar. Anhydrosugar has the structural features resembling that of ethylene glycol; each carbon has one attached hydroxyl group or contains an oxygen function that can be readily converted into a hydroxyl. Ethylene glycol (EG) and propylene glycol (PG) can be produced by selectively cleaving the C—C bonds into C₂ and C₃ units. As such, the presently disclosed subject matter provides a process for the conversion of anhydrosugar feed stock materials and hydrogen gas into glycols, particularly with ethylene glycol as the main product and propylene glycol as a smaller co-product.

The process variables have major impacts on the conversion and selectivity of the reaction. For example, the particular catalyst(s) used and process conditions can provide for a successful reaction selectivity outcome under a set of practical reaction conditions. According to the presently disclosed subject matter, these process variables are identified as being important taking into consideration the chemistry of the reaction discussed below.

The sugars to glycols hydrogenolysis reaction, which is carried out using a metal catalyst and in the presence of hydrogen, is a complex reaction known to produce hundreds of products. Since ethylene and propylene glycols are the desired products, the other products must be minimized by selecting the appropriate bi-functional catalyst system and conditions; additionally an EG/PG wt % ratio of at least 1:1 and preferably 10:1 or more is desirable. Anhydrosugars can undergo hydrolysis to form glucose and other sugar derivatives. In general, glucose and other sugar derivatives tend to cleave into C₃ fragments more easily than the desired C₂ fragment, resulting in the formation of propylene glycol as the single most predominant molecule. While the selection of the most appropriate bi-functional catalyst system, not only from the selectivity point of view but also from the point of view of catalyst longevity, is an important task, other aspects of the reaction must also be considered. The bi-functional catalyst system generally only controls the chemistry taking place on its surface; for example, the cleavage of the glucose and other sugar derivatives into smaller fragments taking place by discrete retro-Aldol reactions followed by hydrogenation of the intermediates into products is the desired pathway. However, quite a number of other reactions take place in solution and these side reactions must also be considered. A number of ions such as OH−, OAc−, etc. could be present in the solution under basic pH conditions or H+ ions could be present under acidic pH conditions. While these ions could also catalyze the retro-Aldol reaction, these ions are generally known to catalyze a variety of dehydration side-reactions causing the glucose and other sugar derivatives to degrade into wasteful products. These undesirable side reactions could become dominant particularly under high temperature conditions. A proper choice of catalysts and process conditions is therefore essential in order to realize the objectives of high glycol yields and long catalyst life. Multiple equations can be used to explain the various steps of the chemistry of the conversion of anhydrosugar to EG and PG, as shown below.

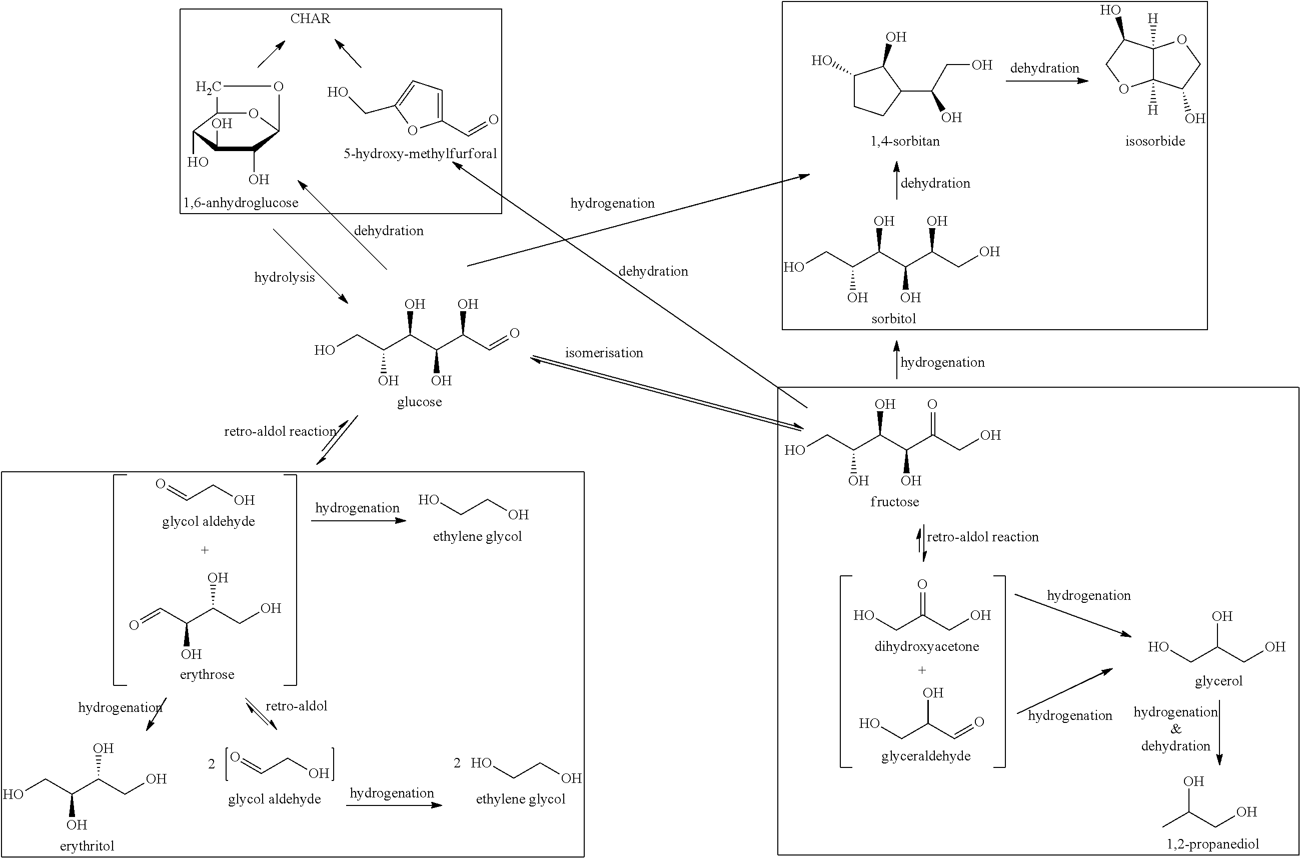

As shown above, the chemistry of anhydrosugars in the hydrogenolysis reaction is a notoriously complex set of functional group chemistries; the products from any reaction could be reactants for all other reactions, including those taking place on the surface of the catalyst. The product distribution (EG, PG, partially converted sugars, etc.) at the end of reaction will be a function of the relative rates of these reactions under the chosen experimental conditions. Thus, according to the presently disclosed subject matter, important process variables have been determined for the disclosed method for producing ethylene glycol from an anhydrosugar feed.

The presently disclosed method for producing ethylene glycol from an anhydrosugar feed has numerous advantages over the prior art. The disclosed method provides for various process conditions that, when combined, achieve superior results in terms of product yield, catalyst stability, and ratio of EG/PG produced. The presently disclosed method allows for the use of an anhydrosugar feed has the advantages of achieving high total glycol yield (i.e., EG and PG), high EG:PG ratio, and having a stable catalyst system.

According to an implementation of the disclosed subject matter, a method for producing ethylene glycol from an anhydrosugar feed may include contacting, in a reactor under hydrogenolysis conditions, the anhydrosugar feed with a bi-functional catalyst system. In an embodiment, the anhydrosugar feed may include at least one of 1,6-anhydro-D-glucose (levogluosan), 1,6-anhydro-D-mannopyranose, 1,6-anhydro-galactopyranose, 1,6-anhydro-D-fructose, 3,6-anhydro-D-glucose, 3,6-anhydro-D-galactose, 1,5-anhydro-D-glucitol, 1,5-anhydro-D-mannitol, 1,5-anhydro-D-xylofuranose, 1,5-anhydro-D-fructose, 2,5-anhydro-D-mannitol, isosorbide, and pyrolytic sugars (pyrolytic molasses).

In embodiments of the present invention, the starting material is suitably an anhydrosugar feedstock comprising at least 1 wt. % anhydrosugar as a solution, suspension or slurry in a solvent. For example, the anhydrosugar feedstock may comprise at least 2 wt. %, at least 5 wt. %, at least 10 wt. %, anhydrosugar in a solvent. Suitably, the anhydrosugar feed comprises a concentration of anhydrosugar, in the total solution entering the reactor, of 1-10 wt % in a solvent.

The solvent present in the reactor is not limited and may be conveniently selected from water, C1 to C6 alcohols, ethers, and other suitable organic compounds, and mixtures thereof. In an embodiment, the solvent is water. If the starting material is provided to the reactor as a solution, suspension or slurry in a solvent, the solvent is also suitably water or a C1 to C6 alcohol, ether, and other suitable organic compounds, and mixtures thereof. In an embodiment, both solvents are the same. In an embodiment, both solvents comprise water.

Suitable reactor vessels to be used in the process of the preparation of ethylene glycol from an anhydrosugar feed include continuous stirred tank reactors (CSTR), plug-flow reactors, slurry reactors, ebullated bed reactors, jet flow reactors, mechanically agitated reactors, back-mixed reactors, bubble columns, such as slurry bubble columns and external recycle loop reactors. The use of these reactor vessels allows dilution of the reaction mixture to an extent that provides high degrees of selectivity to the desired glycol product (mainly ethylene and propylene glycols). There may be one or more of such reactor vessels, arranged in series. In one embodiment, preferably there are two reactor vessels arranged in series, the first one of which is a CSTR, the output of which is supplied into a plug-flow reactor.

The process may be carried out as a batch process or as a continuous flow process.

In one embodiment of the invention, the process is a batch process. In such a process, the reactor may be loaded with the catalyst system, solvent and anhydrosugar, and the reactor may then be pressurized with hydrogen at room temperature, sealed and heated to the reaction temperature.

In embodiments of the invention wherein the process is a batch process, after addition of all of the portions of the starting material, the reaction may then be allowed to proceed to completion for a further period of time. The reaction product will then be removed from the reactor or the reactor product can be sampled in between the additions of the starting material.

In embodiments of the invention wherein the process is carried out as a continuous flow process, after initial loading of some or all of the catalysts and, optionally, solvent, the reactor is heated and pressurized with hydrogen and then the first portion of starting material is introduced into the reactor. Further portions of starting material are then provided to the reactor. Reaction product is removed from the reactor in a continuous manner. In some embodiments of the invention, catalysts may be added in a continuous manner.

The disclosed method for producing ethylene glycol from an anhydrosugar feed may be performed under particular hydrogenolysis conditions in order to maximize the desired yield of EG. For example, the hydrogenolysis conditions may include temperature, pressure, flow rate, and any other process variable that may be controlled. In an embodiment, the temperature in the reactor may be at least 150° C., at least 160° C. and at least 170° C. In an embodiment, the temperature in the reactor may be at most 320° C., at most 300° C., at most 270° C., and at most 250° C. According to an embodiment, the reactor temperature may be in the range of from 150 to 320° C. In an embodiment, the reactor temperature may be in the range of from 170 to 250° C. Preferably, the reactor is heated to a temperature within these limits before addition of any starting material and is maintained at such a temperature until all reaction is complete. The reactor temperature may be increased according to a one-step ramp, a two-step ramp, or a multi-step ramp. In the case of a two-step ramp or multi-step ramp, the reactor contents may be held at the intermediate temperature for a variable duration of time before increasing the temperature to the next step. According to an embodiment, a 2-step temperature ramp may include a temperature in the first step in the range of from 100-150° C., and the temperature in the second step in the range of from 150-320° C.

The pressure in the reactor is generally at least 1 MPa, preferably at least 2 MPa, more preferably at least 3 MPa. The pressure in the reactor is generally at most 25 MPa, more preferably at most 20 MPa, more preferably at most 18 MPa. Preferably, the reactor is pressurized to a pressure within these limits by addition of hydrogen before addition of any starting material and is maintained at such a pressure until all reaction is complete. This can be achieved by subsequent addition of hydrogen.

The process of the present invention may take place in the presence of hydrogen. In an embodiment, the process of the present reaction may take place in the absence of air or oxygen. In order to achieve this, it is preferable that the atmosphere in the reactor be evacuated and replaced with hydrogen repeatedly, after loading of any initial reactor contents, before the reaction starts. It may also be suitable to add further hydrogen to the reactor as the reaction proceeds. In an embodiment, the presently disclosed method may also include contacting the anhydrosugar feed with hydrogen. For example, the disclosed method may take place in the presence of hydrogen. Hydrogen may be supplied into the reactor vessel under pressure in a manner common in the art.

According to an embodiment, the bi-functional catalyst system may include a hydrogenation catalyst, and a retro-Aldol catalyst. In an embodiment, the reactor may be pre-loaded with the hydrogenation catalyst and the retro-Aldol catalyst may be continuously added to the reactor. According to an embodiment, the retro-Aldol catalyst may be continuously added to the reactor via the anhydrosugar feed. The weight ratio of catalyst system to anhydrosugar in the feed is suitably in the range of from 1:100 to 1:10000. In an embodiment, the weight ratio of the retro-Aldol catalytic species suitable for hydrogenation may be in the range of from 0.02:1 to 3000:1, in the range of from 0.1:1 to 100:1, on the basis of the total weight of the catalyst system.

The hydrogenation catalyst may comprise one or more of Ru, Ni, Cu, Co, Pt, Pd, Raney-Ni, Raney-Co, and Raney-Cu. According to an embodiment, the hydrogenation catalyst may include ruthenium. This hydrogenation catalyst may be present in the elemental form or as a compound. It may also be suitable that this hydrogenation catalyst is present in chemical combination with one or more other ingredients in the catalyst system. In an embodiment, the hydrogenation catalyst may be a Raney-type catalyst. According to an embodiment, the hydrogenation catalyst may comprise at least one of Raney-Ni, Raney-Co, Raney-Cu, nano-particle metal. In some cases, the hydrogenation catalyst may be further promoted with one or more metals such as Ni, Fe, Co, Cr, Cu, Mn, Mo, W, Re, Rh, Ru, Pd, Ag, Au, Pt, Ir, and La. In an embodiment, the hydrogenation catalyst may be provided in sulfided form.

The retro-Aldol catalyst may comprise one or more compound, complex or elemental material including silver tungstate, sodium meta-tungstate, ammonium meta-tungstate, sodium poly-tungstate, tungstic acid, alkali- and alkaline-earth metal tungstates, sodium phospho-tungstate, phospho-tungstic acid, alkali- and alkaline-earth metal phospho-tungstates, alkali- and alkaline-earth metal molybdates, alkali- and alkaline-earth metal phospho-molybdates, phospho-molybdic acid, heteropoly acids, mixed tungstates and molybdates, niobic acid, silicotungstic acid, alkali- and alkaline-earth metal niobates, and combinations thereof. The metal component may be in a form other than a carbide, nitride, or phosphide. In an embodiment, the retro-Aldol catalyst composition comprises one or more compound, complex or elemental material selected from those containing tungsten or molybdenum.

According to an embodiment, at least one of the hydrogenation catalyst and retro-Aldol catalyst of the bi-functional catalyst system may be supported on a solid support. In an embodiment, the hydrogenation catalyst and retro-Aldol catalyst may be present in either heterogeneous or homogeneous form. In this case, any other active catalyst component may also be supported on a solid support. In one embodiment, the hydrogenation catalyst is supported on one solid support and the retro-Aldol catalyst is supported on a second solid support which may comprise the same or different material. As a specific example, the hydrogenation catalyst may be a hydrogenation catalyst supported on a hydrothermally stable support. In another embodiment, both the hydrogenation catalyst and retro-Aldol catalyst are supported on one solid hydrothermally stable support.

The solid support may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

According to the presently disclosed subject matter, a product stream may be obtained from the reactor including ethylene glycol and propylene glycol. The product stream may include at least 30 wt %, at least 50 wt %, and at least 70 wt % EG after the removal of water. The product stream may also include 5 wt % of PG after the removal of water. An advantage of the presently disclosed method is the ability to maximize the yield of EG relative to the yield of PG. For example, the product stream may include an EG/PG wt % yield ratio of at least 1:1, a EG/PG wt % yield ratio of at least 7:1, and a EG/PG wt % yield ratio of at least 10:1.

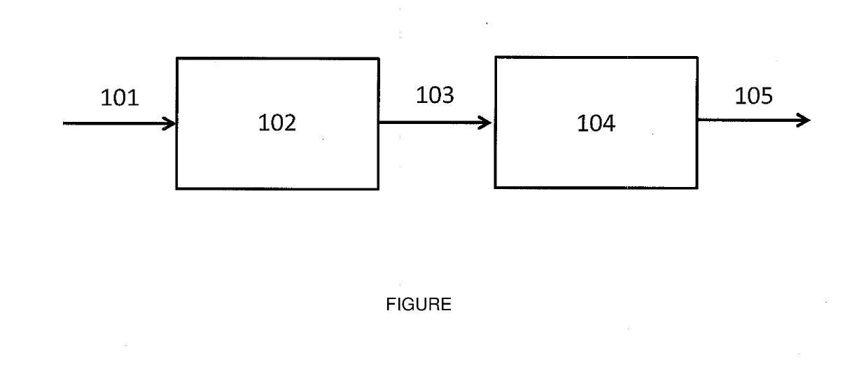

FIG. 1 shows an example process scheme according to an implementation of the disclosed subject matter. As shown in FIG. 1, a feed 101 may include anhydrosugar feed and a solvent and may be provided to a conversion unit 102 to convert it mainly into glucose or other sugar derivatives in solvent to form feed 103. The conversion unit 102 may consist of multiple other units. A feed 103 containing glucose and other sugar derivatives may be fed to the main reactor 104 where it may undergo a reaction in the presence of the bi-catalyst system to produce a product stream comprising of EG and PG 105. Although not shown in FIG. 1, reactor 104 may include an agitator (e.g., magnetic stir bars) for mixing the solution. In one example, the first catalyst with water (e.g., a slurry of the catalyst and water) may be pre-loaded in the reactor 104. The first catalyst may be activated by reduction with hydrogen supplied to the reactor 104. Next, the temperature may be increased to the desired reaction temperature. An additional feed line may be used for feeding the second catalyst into reactor 104. In an example, the reactor 104 may be pre-loaded with the first catalyst and the second catalyst may be continuously added to the reactor 104. In one embodiment, the second catalyst may be continuously added to the reactor 104 via the glucose and other sugar derivatives feed 103. Two or more of the liquid feeds may be combined into one or more feed lines to the reactor 104. In an embodiment, the conversion unit 102 and the reactor 104 may be the same vessel. The pressure in reactor 104 may be controlled by a pressure control valve and excess hydrogen may be vented from reactor 104 via an off-gas line (not shown). A level controlling device (not shown) may measure the volume within reactor 104 in order to maintain a constant volume. The product stream 105 may be removed from reactor 104.

In the disclosed method for the preparation of ethylene glycol from an anhydrosugar-containing feed, the reaction may be run for a time period of up to 6 hours with a stable catalyst system.

As shown in the Examples section provided below, the presently disclosed method for producing ethylene glycol from an anhydrosugar feed has numerous advantages. For example, the process according to the presently disclosed subject matter results in higher selectivity of glycols and conversion as compared to sugars.

### EXAMPLES

### Experimental Apparatus

The apparatus used to perform the experiments shown in Example 1 is described as follows. For the following example, 75 ml Hastelloy C batch autoclaves, with magnetic stir bars, were used to screen various conditions, catalysts and feedstocks. In typical experiments, known weights of catalysts and feedstocks were added to the autoclaves along with 30 ml of the solvent (typically water). If the catalysts or feedstocks were present as slurries or solutions, the total volume of those as well as the solvent was kept at 30 ml.

### Experimental Procedure for Examples 1-5 (1%-2% 1,6-Anhydro-β-D-Glucose Solutions)

The reactions were run in 75 ml Hastelloy C batch autoclaves, with magnetic stir bars, to screen various conditions for the 1%-2% 1,6-anhydro-β-D-glucose solutions in water. In typical experiments, 30 ml of the 1%-2% 1,6-anhydro-β-D-glucose solution was loaded into the autoclave. If less than 30 ml of the feed was available, deionized water was added to make a total liquid volume of 30 ml. Depending on the experiment, known amounts of hydrogenation catalyst and/or retro-Aldol catalysts were also added to the autoclave. The loaded autoclave was then purged three times with nitrogen, followed by hydrogen purge. The pressure was then raised to 2000 psig of hydrogen and the autoclave was sealed and left stirring to perform a leak test.

After performing the leak test, the autoclave was de-pressurized to the target hydrogen pressure at room temperature, and closed. For a one step reaction, the temperature was ramped to 195° C. and held for varying durations including from 75 minutes to 225 minutes. After the last hold, the reactor was cooled to room temperature. A sample of the reactor contents was then taken, filtered and analyzed by High Pressure Liquid Chromatography (HPLC).

Table 1 provides details on the catalyst systems tested and the results obtained in Examples 1-5.

TABLE 1

| | Example 1 | Example 2 | Example 3 | Example 4 | Example 5 |
|---|---|---|---|---|---|
| Feed | 2% 1,6- | 2% 1,6- | 2% 1,6- | 2% 1,6- | 1% 1,6- |
| | anhydroglucose | anhydroglucose | anhydroglucose | anhydroglucose | anhydroglucose |
| H2 pressure | 1450 psig | 1450 psig | 1450 psig | 1450 psig | 1450 psig |
| at room | | | | | |
| temperature | | | | | |
| before | | | | | |
| sealing and | | | | | |
| heating | | | | | |
| RXN | 195 degree C. | 195 degree C. | 195 degree C. | 195 degree C. | 195 degree C. |
| temperature | | | | | |
| RXN time | 225 min | 75 min | 75 min | 135 min | 225 min |
| Cat 1 | Silver | Silver | Sodium | Silver | Silver |
| | tungstate | tungstate | phosphotungstate | tungstate | tungstate |
| Cat 1 amount | 0.0417 g | 0.0417 g | 0.015 g | 0.0417 g | 0.0417 g |
| Cat 2 | 1% Ru on | 1% Ru on | 1% Ru on SiO2 | 1% Ru on | 1% Ru on |
| | SiO2 | SiO2 | | SiO2 | SiO2 |
| Cat 2 amount | 0.125 g | 0.125 g | 0.045 | 0.125 g | 0.125 g |
| Product | | | | | |
| EG | 1.499% | 0.894% | 0.633% | 1.60% | 0.59% |
| PG | 0.051% | n/d | 0.022% | 0.106% | 0.05% |
| 1,6-anhydroglucose | 0.304% | 1.00% | 0.595% | 0.246% | 0% |
| EG Yield | 74.95% | 44.5% | 31.65% | 80% | 59% |
| Conversion | 84.8% | 50% | 70.2% | 87.80% | 100% |
| MEG:MPG | 29:1 | * | 29:1 | 15:1 | 12:1 |

* No formation of MPG was detected by HPLC

As shown in Table 1, examples 1-5 demonstrate high conversions and selectivity to EG according to the presently disclosed subject matter. A more dilute feedstock resulted in higher conversion as seen by comparing Examples 1 and 5. Also, longer duration run lengths resulted in higher conversions of 84.8% and 87.8% as shown in Example 1 (225 minutes) and Example 4 (135 minutes), respectively, as it allows more of the feedstock to breakdown into EG and PG as compared to 70.2% in Example 3 (75 minutes). As demonstrated in Table 1 above, the presently disclosed subject matter provides high selectivity to EG. Specifically, Example 3 provided an EG yield of 31.65%, Example 2 provided an EG yield of 44.5%, and Example 5 provided an EG yield of 59%. Notably, Examples 1 and 4 provided an EG yield of at least 70%, and in particular, 74.95% and 80%, respectively. Also shown in Table 1, the presently disclosed subject matter provides high selectivity to EG and the ratio of EG to PG achieved by all of Examples 1-5 is at least 10:1. In fact, Example 5 resulted in a 12:1 ratio of EG to PG, Example 4 resulted in a 15:1 ratio of EG to PG, Examples 1 and 3 both resulted in a 29:1 ratio of EG to PG, and Example 2 resulted in no PG detected. This further demonstrates the selectivity to EG achieved by the presently disclosed subject matter. Further, as shown in these Examples 1-5, various tungsten-based catalysts can be used effectively to achieve relatively high yields of glycols.

The foregoing description, for purpose of explanation, has been described with reference to specific embodiments. However, the illustrative discussions above are not intended to be exhaustive or to limit embodiments of the disclosed subject matter to the precise forms disclosed. Many modifications and variations are possible in view of the above teachings. The embodiments were chosen and described in order to explain the principles of embodiments of the disclosed subject matter and their practical applications, to thereby enable others skilled in the art to utilize those embodiments as well as various embodiments with various modifications as may be suited to the particular use contemplated.

What is claimed is:

1. A method for producing glycols from an anhydrosugar feed comprising:
a) contacting, in a reactor under hydrogenolysis conditions, the anhydrosugar feed with a bi-functional catalyst system comprising:
1) a retro-Aldol catalyst, and
2) a hydrogenation catalyst;
b) obtaining a product stream, from the reactor, comprising ethylene glycol and propylene glycol.

2. The method of claim 1, wherein the anhydrosugar feed comprises a concentration of anhydrosugar, in the total solution entering the reactor, of 1-10 wt % in a solvent.

3. The method of claim 1, wherein the anhydrosugar feed is derived from at least one selected from the group consisting of: dehydration of sugars or isolated from fast pyrolysis oil (bio oil).

4. The method of claim 2, wherein the solvent is H₂O.

5. The method of claim 1, wherein the anhydrosugar feed comprises at least one selected from the group consisting of: 1,6-anhydro-D-glucose, 1,6-anhydro-D-mannopyranose, 1,6-anhydro-galactopyranose, 1,6-anhydro-D-fructose, 3,6-anhydro-D-glucose, 3,6-anhydro-D-galactose, 1,5-anhydro-D-glucitol, 1,5-anhydro-D-mannitol, 1,5-anhydro-D-xylofuranose, 1,5-anhydro-D-fructose, 2,5-anhydro-D-mannitol, isosorbide, and pyrolytic sugars.

6. The method of claim 1, wherein the hydrogenolysis conditions comprise a temperature in the range of from 150-320° C.

7. The method of claim 1, wherein the hydrogenolysis conditions comprise a temperature in the range of from 170-250° C.

8. The method of claim 1, wherein the hydrogenolysis conditions comprise a 2-step temperature ramp, wherein the temperature in the first step is in the range of from 100-150° C., and the temperature in the second step is in the range of from 150-320° C.

9. The method of claim 1, wherein the reactor is pre-loaded with the hydrogenation catalyst and the retro-Aldol catalyst is continuously added to the reactor.

10. The method of claim 9, wherein the retro-Aldol catalyst is continuously added to the reactor via the anhydrosugar feed.

11. The method of claim 1, wherein the hydrogenation catalyst comprises at least one selected from the group consisting of: Ru, Ni, Cu, Co, Pt, Pd, Raney-Ni, Raney-Co, and Raney-Cu.

12. The method of claim 1, wherein the hydrogenation catalyst comprises ruthenium.

13. The method of claim 11, wherein the hydrogenation catalyst is further promoted with one or more selected from the group consisting of: Ni, Fe, Co, Cr, Cu, Mn, Mo, W, Re, Rh, Ru, Pd, Ag, Au, Pt, Ir, and La.

14. The method of claim 1, wherein the retro-Aldol catalyst comprises at least one selected from the group consisting of: silver tungstate, sodium meta-tungstate, ammonium meta-tungstate, sodium poly-tungstate, tungstic acid, alkali- and alkaline-earth metal tungstates, sodium phospho-tungstate, phospho-tungstic acid, alkali- and alkaline-earth metal phospho-tungstates, alkali- and alkaline-earth metal molybdates, alkali- and alkaline-earth metal phospho-molybdates, phospho-molybdic acid, heteropoly acids, mixed tungstates and molybdates, niobic acid, silicotungstic acid, alkali- and alkaline-earth metal niobates.

15. The method of claim 1, wherein the retro-Aldol catalyst is a heterogeneous catalyst.

16. The method of claim 1, wherein the retro-Aldol catalyst is a homogeneous catalyst.

17. The method of claim 1, wherein the product stream comprises a yield of at least 30 wt % EG.

18. The method of claim 1, wherein the product stream comprises an EG/PG wt % yield ratio of at least 10:1.
